(19) United States
(12) Patent Application Publication
(10) Pub. No.: US 2005/0255132 A1
(43) Pub. Date: Nov. 17, 2005

(54) Softening cream

(76) Inventor: Tali Rabiner, Ganei-Tikva (IL)

Correspondence Address: Eitan Law Group;LandonIP Inc, Suitte 450, 1700 Diagonal Road, New York, NY, 22314, US

(21) Appl. No.: 10/497,391

(22) PCT Filed:
(86) PCT No.: PCT/IL02/00968

Related U.S. Application Data
(60) Provisional application No. 60/334,596, filed on Dec. 3, 2001.

(51) Int. Cl. A61K 35/78; A61K 7/06; A61K 7/11

(52) U.S. Cl. 424/401; 424/70.13; 424/771

(57) ABSTRACT

The present invention relates to a composition for softening skin comprising an effective amount of a salicylic acid embedded in starch microcapsules and an antifungal compound. The present invention further relates to a method of treating the skin to reduce wrinkling, modifying its color, reducing surface pigmented spots or eliminating squamae. The present invention further relates to a method of manufacturing a skin softening cream.

### FIELD OF THE INVENTION

The present invention relates to preparations and methods of use thereof for pharmaceutical and cosmetic treatment of skin.

### BACKGROUND OF THE INVENTION

In the course of normal aging, human skin undergoes degenerative changes including drying, wrinkling, reduced elasticity, changes in connective tissue matrix components, denudation, colour changes, roughening of texture, increased resistance to pressure, thickening, reduced moisture content, increased hardness, or a combination thereof. Skin aging is most often perceived as undesirable and, in anticipation of aging or as a result of getting older, both men and women seek medical or cosmetic remedies in order to treat their aging skin. For this purpose, a wide variety of methods and products are available, as may easily be determined by visiting any local chemist or pharmacy. Thus, a common form treating skin is through the topical application of agents that serve to modify one or more characteristics associated with the aging process.

During the aging process, the appearance on the skin of different signs very characteristic of aging, reflected, in particular, in a modification of skin structure and function, is observed. These signs are especially pronounced on uncovered areas such as face and hands, on which special characteristics due to exposure of the skin to sunlight (actinic aging) are generally added.

On an uncovered area, it is difficult, in general, to distinguish clearly between signs originating from the physiological process of skin aging and signs due to repeated exposure of the skin to light.

The main clinical signs of skin aging are the following: hardening, drying, wrinkling, disorganization of the micro-relief pattern or grain of the skin, decoloration, pallor, yellowing, loss of local microcirculation, disorganization of local microcirculation, hyperpigmentation, or a combination thereof. Other signs of skin hardening are a dry and rough appearance of the skin, which is due chiefly to greater desquamation, these squamae contributing also to the somewhat gray appearance of the color by diffracting light rays. Finally, a loss is noted in firmness and tonus of the skin, which, as in the case of wrinkles, is explained at least partially by a dermal and epidermal atrophy as well as a flattening of the dermoepidermal formation.

It is hence noted that the clinical signs of skin aging result chiefly from a dysfunction of the main biological mechanisms operating in the skin.

Hard skin feels taut and dry; may have patches of flaking and visible dehydration; is particularly prone to fine lines and wrinkles. Thus, among the treatment goals are to replenish moisture; protect against on-going loss of moisture; encourage the skin's natural moisturizing factors to function longer; protect against free radicals, ultraviolet radiation and other causes of premature wrinkling.

In administrating a keratolytic agent, the aim is to keep skin soft by avoiding cell build-up. A keratolytic agent encourages exfoliation, i.e. removal of dead skin cells, and is a crucial step in any slain care program. In one mechanism of skin hardening, dead skin cells are not properly removed, accumulation of dead tissue occurs and the stratum corneum layer thickens. The result is an appearance of hardening and aging including, but not only, color changes, wrinkling, uneven texture, blemishing, flaking, roughening, hardness to the touch, and loss of appeal. Furthermore, the thick layer of keratinized cells forms a barrier, preventing products that might help alleviate these conditions from reaching the deeper epidermal layers.

The salicylates are a chemical family found in plants, in particular the willow tree, wintergreen leaves, sweet birch, and having a wide variety of pharmacological properties including, inter alia, anti-inflammatory, anti-phlogistic, anti-thrombotic actions, anti-psoriatic, keratolytic, exfoliative, anti-seborrhea, and anti-microbial.

Salicylic acid is known as a micro-exfolient that improves the appearance and condition of the skin. Its action causes a removal of the surface cells that are already old, thereby forcing the germination layers to push new cells to the surface of the skin. The newer cells can then rebuild an adequate defense barrier against damaging external agents. The skin becomes smoother and more compact, with an improved texture and translucence. Sodium salicylate (the sodium salt of salicylic acid) is often used in products because it dissolves better in water and gels. When this salt comes in contact with the skin, it interacts with the acid mantle of the skin and forms free salicylic acid.

However, in the treatment of skin conditions, salicylic acid has been used with only limited success and it appears that the delivery systems for use of salicylate-based compounds have not been optimized with respect to the treatment of skin. Furthermore, while salicylates penetrate into the skin, they tend to irritate the skin and may trigger symptoms of inflammation. The present invention solves this problem through providing compositions and methods of use thereof wherein the salicylate ingredient is applied as a non-irritant and sustained release formulation suitable for topical application to virtually any area of the skin and for multiple and prolonged periods of time.

### SUMMARY OF THE INVENTION

In one embodiment, the present invention provides a composition for softening skin comprising a salicylate embedded in starch microcapsules in combination with an antifungal compound such as willow bark extract.

In another embodiment, the present invention provides a composition in a vehicle suitable for topical application.

In another embodiment, the present invention provides a composition is a cream, a gel, an ointment, a topical lotion, a lotion, a milk, a stick, a foam, an oil, a soap, a fatty composition, a paste or pressurized in an aerosol device.

In one embodiment, the present invention provides a composition wherein the antifungal compound is a willow bark extract.

In one embodiment, the composition of the present invention further comprises one or more of an emollient a lubricant, a moisturizer, a protectant, an enhancer of skin penetration, a softener, a pH adjuster, a cooling agent, a solubilizer, a preservative or perfume agent.

In one embodiment, the present invention provides a composition wherein the emollient is stearic acid, cetyl alcohol or paraffin liquid.

In one embodiment, the present invention provides a composition wherein the lubricant is cetyl alcohol, paraffin liquid, stearic acid or cocoa butter.

In one embodiment, the present invention provides a composition wherein the moisturizer is propylene glycol or dimethicone.

In one embodiment, the present invention provides a composition wherein the protectant is propylene glycol or dimethicone.

In one embodiment, the present invention provides a composition wherein the enhancer of skin penetration is propylene glycol or dimethicone.

In one embodiment, the present invention provides a composition wherein the softener is cocoa butter.

In one embodiment, the present invention provides a composition wherein the pH adjuster is triethanolamine.

In one embodiment, the present invention provides a composition wherein the cooling agent is menthyl lactate.

In one embodiment, the present invention provides a composition wherein the solubilizer is polysorbate 60.

In one embodiment, the present invention provides a composition wherein the preservative is imidazolidinyl urea, triclosan, methyl paraben or propyl paraben.

In one embodiment, the present invention provides a composition wherein the perfume agent is fragrance.

In one embodiment, the present invention provides a composition suitable for topical application to human skin. In another embodiment, the composition is a cream. In another embodiment, the cream is a skin softener.

In one embodiment, the present invention provides a composition comprising salicylic acid (10%) embedded in starch microcapsules in a percentage ranging from 15 to 25 and a willow bark extract in a percentage ranging from 3 to 6.

In one embodiment, the present invention provides a composition wherein the steric acid is in a percentage ranging from 3 to 6.

In one embodiment, the present invention provides a composition wherein the cetyl alcohol is in a percentage ranging from 2.5 to 5.

In one embodiment, the present invention provides a composition wherein the paraffin liquid is in a percentage ranging from 2 to 4.

In one embodiment, the present invention provides a composition wherein the cocoa butter is in a percentage ranging from 1 to 3.

In one embodiment, the present invention provides a composition wherein the propylene glycol is in a percentage ranging from 2 to 4.

In one embodiment, the present invention provides a composition wherein the dimethicone is in a percentage ranging from 1 to 3.

In one embodiment, the present invention provides a composition wherein the triethanolamine is in a percentage ranging from 1 to 3.

In one embodiment, the present invention provides a composition wherein the menthyl lactate is in a percentage ranging from 0.8 to 1.5.

In one embodiment, the present invention provides a composition wherein the polysorbate is in a percentage ranging from 0.8 to 1.6.

In one embodiment, the present invention provides a composition wherein the imidazolidinyl urea is in a percentage ranging from 0.3 to 0.5.

In one embodiment, the present invention provides a composition wherein the triclosan is in a percentage ranging from 0.2 to 0.3.

In one embodiment, the present invention provides a composition wherein the methyl paraben is in a percentage ranging from 0.05 to 0.15.

In one embodiment, the present invention provides a composition wherein the propyl paraben is in a percentage ranging from 0.05 to 0.15.

In one embodiment, the present invention provides a composition wherein the fragrance is in a percentage ranging from 0.3 to 0.6.

In one embodiment, the present invention provides a method of treating the skin to reduce wrinkling, modify its color, reduce surface pigmented spots or eliminate squamae, comprising applying to the skin in an effective amount of the composition according to the invention.

In one embodiment, the present invention provides a method of softening skin, comprising applying to the skin an effective amount of a composition according to the invention.

In one embodiment, the present invention provides a method of treating a subject having hardening skin, comprising the step of administering to the skin of the subject an amount of a composition according to the invention

In one embodiment, the present invention provides a method wherein the composition is applied to the skin in an amount ranging from 1 to 10 mg per cm² of skin for a period of time ranging from one week to about one year.

In one embodiment, the present invention provides a cosmetic device comprising an effective amount of a composition according to the invention.

In one embodiment, the present invention provides a pharmaceutical preparation comprising an effective amount of a composition according to the invention.

The present invention further provides a method of manufacturing a skin softening cream, comprising the following steps:
heating stearic acid, cetyl alcohol, paraffin liquid, dimethicone and cocoa butter to form a hydrophobic mixture until the hydrophobic mixture reaches 75° C.;
heating willow bark extract, propylene glycol, triethanolamine, polysorbate 60, aloe vera dry powder 1:200 and distilled water to form a hydrophilic mixture until the hydrophilic mixture reaches 75° C.;
mixing/blending the hydrophobic and hydrophilic mixtures using a vacuum mixer to form a third mixture;
emulsifying the third mixture using homogenizer for 10 min to form an emulsion;
cooling the emulsion by mixing until the emulsion reaches 45° C.;
adding starch microcapsules containing salicylic acid, imidazolidinyl urea, methyl paraben, propyl paraben and fragrance into the emulsion to form a final mixture; and
cooling the final mixture by continues mixing until the final mixture reaches 35° C.

### DETAILED DESCRIPTION OF THE INVENTION

The present invention provides a composition for softening skin comprising a salicylate embedded in starch microcapsules in combination with an antifungal compound such as willow bark extract. The present invention further provides a method of manufacturing a skin softening cream, comprising the following steps: heating stearic acid, cetyl alcohol, paraffin liquid, dimethicone and cocoa butter to form a hydrophobic mixture until the hydrophobic mixture reaches 75° C., heating willow bark extract, propylene glycol, triethanolamine, polysorbate 60, aloe vera dry powder 1:200 and distilled water to form a hydrophilic mixture until the hydrophilic mixture reaches 75° C., mixing/blending the hydrophobic and hydrophilic mixtures using a vacuum mixer to form a third mixture, emulsifying the third mixture using homogenizer for 10 min to form an emulsion, cooling the emulsion by mixing until the emulsion reaches 45° C., adding starch microcapsules containing salicylic acid, imidazolidinyl urea, methyl paraben, propyl paraben and fragrance into the emulsion to form a final mixture and cooling the final mixture by continues mixing until the final mixture reaches 35° C.

The present invention provides a composition for softening skin, wherein the composition comprises a salicylate embedded in starch microcapsules in combination with an antifungal compound such as willow bark extract.

The applicant discovered, surprisingly, that the use of salicylic acid derivatives embedded in starch microcapsules enabled the skin hardening to be retarded, alleviated, or a combination thereof, and the clinical signs of aging to be effected in a similar manner, without producing skin irritation or sensations of discomfort. Applicant found, more especially, that, by means of the composition according to the invention, it is possible to reduce inkling at least, to modify the color of the skin, which appears more pinkish, to obliterate surface pigmented spots, to eliminate squamae and to give a more elastic consistency to the skin. It was found, in particular, that it was possible to impart a much softer feel to the skin.

Starch microcapsules containing salicylic acid are known per se, although they have not been known to be useful in combination with other components, such as disclosed herein, to form a composition which is a skin softener. Starch microcapsules have been suggested for use in deodorants. One advantage of starch microcapsules containing a therapeutic agent is in the controlled or sustained release of the agent over time. According to the present invention, the therapeutic agent contained within the starch microcapsules is a salicylate or pharmaceutically acceptable derivative thereof. In one embodiment of the invention, the salicylate is salicylic acid. Although salicylic acid has been used in the topical treatment of skin conditions, such as acne, a composition comprising a salicylate embedded in microcapsules in combination with one or more dermatological agents including one or more of an anti fungal compound, an emollient, a lubricant, a moisturizer, a protectant, an enhancer of skin penetration, a softener, a pH adjuster, a cooling agent, a solubilizer, a preservative or perfume agent, has not been known to be of therapeutic value or to have other advantages of the present invention.

The present invention provides a composition and a method of treating the skin comprising microcapsules of salicylic acid modified through combination with a system for topical administration. The composition of the invention is intended for use as a pharmaceutical or cosmetic preparation in the topical treatment of skin, wherein it is desired to optimize the delivery of a salicylate upon topical application. Such a composition has excellent properties when used as a keratolytic or a skin softener, wherein the composition comprises a salicylate embedded in starch microcapsules in combination with one or more dermatological agents including an emollient, a lubricant, a moisturizer, a protectant, an enhancer of skin penetration, a softener, a pH adjuster, a cooling agent, a solubilizer, a preservative or perfume agent. The composition can also contain any other ingredient customarily used in compositions intended for topical application.

In the treatment of hardening skin, a skin softener is applied to the affected area in the form of a gel, cream, milk stick foam, soap, oil, fatty composition, lotion, a paste, or pressurized in an aerosol device.

Skin hardening is typically a part of the aging process, thus skin softeners are preferably applied to the skin throughout the life cycle of the individual. Particularly preferred is to treat hardening skin beginning in the second half of the individual's lifetime, as the effects of skin aging, including hardening, become more prominent. A frequent mode of treating hardening skin is to begin treatment with a skin softener on a daily basis when the initial or premonitory signs of skin hardening first become apparent. Since areas of the skin exposed to light and pressure are particularly prone to hardening, the compositions of the invention are applied, in one embodiment of the invention, to the limbs, face, or feet, on the basis of 1 to 10 mg of composition per cm² of skin, for a period which can range from one week to one year or longer.

In one embodiment of the invention, the composition is suitable for the topical treatment of skin, in particular for the treatment of hard skin, whereupon it is desirable to obtain a softer texture of one or more layers of the skin.

In another embodiment of the invention, the composition is suitable for the chronic treatment of hardening skin, wherein the composition is administered over a period of months or years.

In another embodiment of the invention, the composition is suitable for the chronic treatment of hardening skin, wherein the composition is administered at least once per day.

According to the present invention, the composition is preferably administered in the form of a gel, cream, milk, stick, foam, soap, oil, fatty composition, lotion, a paste, or pressurized in an aerosol device, and may also contain any other ingredient customarily used in compositions intended for topical application. Similarly, the composition of the present invention may be applied alone, or in combination with mechanical exfolients including, but not only, sponges, abrasive surfaces, abrasive particles, clothes, peels, soaps, or a combination thereof.

Salicylic acid embedded in starch microcapsules are prepared for example using submicrodispersion technology, microfluidizing techniques, or a combination thereof. For the purpose of implementing the present invention, salicylic acid embedded in starch microcapsules, known as well as starch microcapsules containing salicylic acid, may contain from 1 to 20% by weight of salicylic acid. The starch of said microcapsules may be obtained from any source containing a starch, which is suitable in either raw or processed form, to form microcapsules containing a salicylic acid. The starch microcapsules containing a salicylic acid may, according to the present invention, comprise other ingredients, in particular organic solvents, or a combination thereof. Examples of said organic solvents are ethanol, methanol, propanol, isopropanol, or a combination thereof. Starch microcapsules containing salicylic acid may be obtained commercially, for example, from LIPOTEC, S.A., product code E21L96.

In one embodiment, the composition of the invention comprises a salicylic acid embedded in starch microcapsules, willow bark extract, stearic acid, cetyl alcohol, liquid paraffin, propylene glycol, dimethicone, cocoa butter, triethanolamine, menthyl lactate, polysorbate 60, imidazolidinyl urea, triclosan, methyl paraben, propyl paraben, fragrance, and water.

In another embodiment of the composition of the invention, said composition is suitable for topical application to human skin.

In another embodiment of the composition of the invention, said composition is a cream.

In one embodiment of the cream of the invention, said cream is a skin softener.

In one particular embodiment of the composition of the invention, said composition comprises: salicylic acid (10%) embedded in starch microcapsules, in a percentage ranging from 15 to 25; willow bark extract, in a percentage ranging from 3 to 6; stearic acid, in a percentage ranging from 3 to 6; cetyl alcohol, in a percentage ranging from 2.5 to 5; liquid paraffin, in a percentage ranging from 2 to 4; propylene glycol, in a percentage ranging from 2 to 4; dimethicone, in a percentage ranging from 1 to 3; cocoa butter, in a percentage ranging from 1 to 3; triethanolamine, in a percentage ranging from 1 to 3; menthyl lactate, in a percentage ranging from 0.8 to 1.5; polysorbate 60, in a percentage ranging from 0.8 to 1.6; imidazolidinyl urea, in a percentage ranging from 0.3 to 0.5; triclosan, in a percentage ranging from 0.2 to 0.3; methyl paraben, in a percentage ranging from 0.05 to 0.15; propyl paraben, in a percentage ranging from 0.05 to 0.15; fragrance, in a percentage ranging from 0.3 to 0.6; and water, to 100%.

In another particular embodiment of the composition of the invention, said composition comprises:

| CTFA Name | Percentage |
|---|---|
| 1. Distilled water | To 100 |
| 2. Starch Microcapsules | 15.00 |
| containing Salicylic Acid | |
| (10% in mixture) with | |
| Alcohol SDA 40 | |
| 3. Willow bark extract | 3.00 |
| 4. Stearic acid | 3.00 |
| 5. Cetyl alcohol | 2.50 |
| 6. Paraffin Liquid | 2.00 |
| 7. Propylene Glycol | 2.00 |
| 8. Dimethicone | 1.00 |
| 9. Cocoa Butter | 1.00 |
| 10. Triethanolamine | 1.00 |
| 11. Menthyl Lactate | 0.80 |
| 12. Polysorbate 60 | 0.80 |
| 13. Imidazolidinyl Urea | 0.30 |
| 14. Triclosan | 0.20 |
| 15. Methyl Paraben | 0.10 |
| 16. Propyl Paraben | 0.10 |
| 17. Fragrance | 0.50 |

### EXAMPLES

The examples that follow are designed to illustrate the invention without, however, being restrictive in nature.

### Example 1

The following composition was prepared in the form of a gel:

| CTFA Name | Percentage |
|---|---|
| 1. Distilled water | To 100 |
| 2. Starch Microcapsules | 15.00 |
| containing Salicylic Acid | |
| (10% in mixture) with | |
| Alcohol SDA 40 | |
| 3. Willow bark extract | 3.00 |
| 4. Stearic acid | 3.00 |
| 5. Cetyl alcohol | 2.50 |
| 6. Paraffin Liquid | 2.00 |
| 7. Propylene Glycol | 2.00 |
| 8. Dimethicone | 1.00 |
| 9. Cocoa Butter | 1.00 |
| 10. Triethanolamine | 1.00 |
| 12. Polysorbate 60 | 0.80 |
| 13. Imidazolidinyl Urea | 0.30 |
| 15. Methyl Paraben | 0.10 |
| 16. Propyl Paraben | 0.10 |
| 17. Fragrance | 0.50 |
| 18. Aloe Vera Dry powder | 0.035 |
| 1:200 | |

The following composition was prepared in the form of a cream:

| CTFA Name | Percentage |
|---|---|
| 1. Distilled water | To 100 |
| 2. Starch Microcapsules | 5.00 |
| containing Salicylic Acid | |
| (10% in mixture) with | |
| Alcohol SDA 40 | |
| 3. Willow bark extract | 3.00 |
| 4. Stearic acid | 6.50 |
| 5. Cetyl alcohol | 3.00 |
| 6. Paraffin Liquid | 3.00 |
| 7. Cera Alba | 2.50 |
| 8. Dimethicone | 2.00 |
| 9. Isopropyl hyristate | 2.50 |
| 10. Triethanolamine | 2.50 |
| 11. Menthyl Lactate | 0.80 |
| 12. Alcohol | 0.80 |
| 13. Lanolin | 1.50 |
| 14. Imidazolidinyl Urea | 0.20 |
| 15. Triclosan | 0.20 |
| 16. Methyl Paraben | 0.10 |
| 17. Propyl Paraben | 0.05 |
| 18. Fragrance | 0.50 |
| 19. Glycerin | 10.00 |

### Example 2

The scotch Tape Test:

The keratolytic effect of the composition of the invention was demonstrated in the scotch tape test. Following treatment with the composition, the accumulation of dead cells at the site of treatment on one foot of a subject was compared to an equivalent, but untreated site on the other foot (control area). A piece of clear scotch tape was adhered to the areas, peeled off and examined for cellular debris. The treated area had significantly less cell debris than the control area according to the scotch tape test.

1. A composition for softening skin comprising all effective amount of a salicylic acid embedded in starch microcapsules and an antifungal compound.

2. A composition according to claim 1, wherein said composition is in a vehicle suitable for topical application.

3. A composition according to claim 1, wherein said composition is a cream, a gel, an ointment, a topical lotion, a lotion, a milk, a stick, a foam, an oil, a soap, a fatty composition, a paste or pressurized in an aerosol device.

4. The composition of claim 1 wherein said antifungal compound is a willow bark extract.

5. The composition according to claim 1 further comprising one or more of an emollient, a lubricant, a moisturizer, a protectant, an enhancer of skin penetration, a softener, a pH adjuster, a cooling agent, a solubilizer, a preservative or perfume agent.

6. The composition of claim 5 wherein said emollient is stearic acid, cetyl alcohol or paraffin liquid.

7. The composition of claim 5 wherein said lubricant is cetyl alcohol, paraffin liquid, stearic acid or cocoa butter.

8. The composition of claim 5 wherein said moisturizer is propylene glycol or dimethicone.

9. The composition of claim 3 wherein said protectant is propylene glycol or dimethicone.

10. The composition of claim 5 wherein said enhancer of skin penetration is propylene glycol or dimethicone.

11. The composition of claim 5 wherein said softener is cocoa butter.

12. The composition of claim 5 wherein said pH adjuster is triethanolamine.

13. The composition of claim 5 wherein said cooling agent is menthyl lactate.

14. The composition of claim 5 wherein said solubilizer is polysorbate 60.

15. The composition of claim 5 wherein said preservative is imidazolidinyl urea, triclosan, methyl paraben or propyl paraben.

16. The composition of claim 5 wherein said perfume agent is fragrance.

17. The composition of claim 1 wherein said composition is suitable for topical application to human skin.

18. The composition of claim 17 wherein said composition is a cream.

19. The composition of claim 18 wherein said cream is a skin softener.

20. The composition of claim 4 wherein said composition comprises salicylic acid (10%) embedded in starch microcapsules in a percentage ranging from 15 to 25 and a willow bark extract in a percentage ranging from 3 to 6.

21. The composition according to claim 6 wherein the stearic acid is in a percentage ranging from 3 to 6.

22. The composition according to claim 6 wherein the cetyl alcohol is in a percentage ranging from 2.5 to 5.

23. The composition according to claim 6 wherein the paraffin liquid is in a percentage ranging from 2 to 4.

24. The composition according to claim 7 wherein the cocoa butter is in a percentage ranging from 1 to 3.

25. The composition according to claim 8 wherein the propylene glycol is in a percentage ranging from 2 to 4.

26. The composition according to claim 8 wherein the dimethicone is in a percentage ranging from 1 to 3.

27. The composition according to claim 12 wherein the triethanol amine is in a percentage ranging from 1 to 3.

28. The composition according to claim 13 wherein the menthyl lactate is in a percentage ranging from 0.8 to 1.5.

29. The composition according to claim 14 wherein the polysorbate is in a percentage ranging from 0.8 to 1.6.

30. The composition according to claim 15 wherein the imidazolidinyl urea is in a percentage ranging from 0.3 to 0.5

31. The composition according to claim 15 wherein the triclosan is in a percentage ranging from 0.2 to 0.3.

32. The composition according to claim 15 wherein the methyl paraben is in a percentage ranging from 0.05 to 0.15.

33. The composition according to claim 15 wherein the propyl paraben is in a percentage ranging from 0.05 to 0.15.

34. The composition according to claim 16 wherein the fragrance is in a percentage ranging from 0.3 to 0.6.

35. A method of treating the skin to reduce wrinkling, modifying color, reducing surface pigmented spots or eliminating squamae, comprising the step of applying to the skin an effective amount of a composition according to claim 1.

36. A method of softening skin, comprising the step of applying to the skin an effective amount of a composition according to claim 1.

37. A method of treating a subject having hardening skin, comprising the step of administering to the skin of said subject an amount of a composition according to claim 1, thereby treating said hardening skin.

38. The method according to claim 35 wherein said composition is applied to the skin in an amount ranging from 1 to 10 mg per cm² of skin for a period of time ranging from one week to about one year.

39. A cosmetic device comprising an effective amount of a composition according to claim 1.

40. A pharmaceutical preparation comprising an effective amount of a composition according to claim 1.

41. A method of manufacturing a skin softening cream, comprising:
heating stearic acid, cetyl alcohol, paraffin liquid, dimethicone and cocoa butter to form a hydrophobic mixture until said hydrophobic mixture reaches 75° C.;
heating willow bark extract, propylene glycol, triethanolamine, polysorbate 60, aloe vera dry powder 1:200 and distilled water to form a hydrophilic mixture until said hydrophilic mixture reaches 75° C.;
mixing/blending said hydrophobic and hydrophilic mixtures using a vacuum mixer to form a third mixture;
emulsifying said third mixture using homogenizer for 10 min to form an emulsion;
cooling said emulsion by mixing until said emulsion reaches 45° C.;
adding starch microcapsules containing salicylic acid, imidazolidinyl urea, methyl paraben, propyl paraben and fragrance into said emulsion to form a final mixture; and
cooling said final mixture by continuous mixing until said final mixture reaches 35° C.
